(19) United States
(12) Patent Application Publication
(10) Pub. No.: US 2026/0272816 A1
(43) Pub. Date: Sep. 17, 2026

(54) SILICONE-FREE SHAMPOO COMPOSITION

(71) Applicant: THE PROCTER & GAMBLE COMPANY, Cincinnati, OH (US)

(72) Inventor: Dariush HOSSEINPOUR, Mason, OH (US)

(21) Appl. No.: 19/558,727

(22) Filed: Mar. 6, 2026

Related U.S. Application Data
(60) Provisional application No. 63/770,470, filed on Mar. 12, 2025.

(51) Int. Cl. A61K 8/92; A61K 8/37; A61K 8/44; A61K 8/86; A61Q 5/02

(52) U.S. Cl. CPC A61K 8/922; A61K 8/375; A61K 8/44; A61K 8/86; A61Q 5/02; A61K 2800/33; A61K 2800/34; A61K 2800/48

(57) ABSTRACT

A shampoo composition comprises a surfactant system comprising an anionic surfactant; a conditioning agent comprising a botanical oil; about 0.5 wt. % to about 3 wt. % of an associative thickener; and a liquid carrier. The associative thickener can be a hydrophobically modified polyether. The composition can be substantially silicone-free.

### CROSS REFERENCE TO RELATED APPLICATIONS

This application claims the benefit, under 35 U.S.C. § 119 (e), to U.S. Provisional Application No. 63/770,470 filed Mar. 12, 2025, the entire disclosure of which is fully incorporated by reference herein.

### FIELD OF THE INVENTION

The present invention generally relates to shampoo compositions that are free of silicone, and more specifically to shampoo compositions that contain an anionic surfactant, an associative thickener, and a conditioning agent comprising a botanical oil that provides similar conditioning performance as compared to shampoos that contain silicone.

### BACKGROUND OF THE INVENTION

Human hair becomes soiled due to its contact with the surrounding environment and from the sebum secreted by the scalp, in addition to the use of third-step stylers and hair treatments that leave a visually or tactilely noticeable residue behind on the hair. The soiling of hair causes it to have a dirty feel and an unattractive appearance, necessitating regular shampooing.

Shampooing cleans the hair by removing excess soil and sebum. However, shampooing can leave the hair in a wet, tangled, and generally unmanageable state. Once the hair dries, it is often left in a dry, rough, lusterless, and/or frizzy condition due to removal of the hair's botanical oils. Therefore, shampoos that provide a cleansing and conditioning benefit to hair (hereinafter “conditioning shampoos”) are popular. Conditioning shampoos frequently contain silicones that coat the hair shaft, thereby locking in moisture, which reduces frizz and gives hair a coveted soft and silky feel. Silicones can provide these conditioning benefits while not interfering with cleansing efficacy.

However, some consumers prefer hair care products that are silicone-free. Many alternatives, such as botanical oils, have been incorporated into shampoo compositions. However, these alternatives are generally less effective and/or leave the hair looking and feeling greasy and/or dirty.

Therefore, there is a need for a silicone-free shampoo composition that provides good conditioning and cleaning benefits.

### SUMMARY OF THE INVENTION

A shampoo composition comprising: (a) a surfactant system comprising an anionic surfactant; (b) a conditioning agent comprising a botanical oil; (c) about 0.5 wt. % to about 3 wt. % of an associative thickener; and (d) a liquid carrier. The composition is substantially free of silicone.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is a chart showing viscosity as a function of associative thickener content for Example 1.

FIG. 2 is a bar graph showing the average detangling force in gram force (gf) values for wet hair in Example 2.

FIG. 3 is a bar graph showing the average detangling force in gram force (gf) values for dry hair in Example 2.

FIG. 4 is a chart showing viscosity as a function of associative thickener content for Example 3.

### DETAILED DESCRIPTION OF THE INVENTION

Shampoo compositions comprising various combinations of anionic surfactants and hair conditioning agents are known. Many of these products comprise anionic surfactant, cationic polymer, and silicone that combine to form coacervate, which deposits on hair during the wash, ultimately giving the hair a smooth texture and clean, silky feel.

However, some consumers want a liquid conditioning shampoo that is silicone-free. It is difficult to formulate a consumer-acceptable conditioning shampoo that is silicone-free because it is a very effective hair conditioning agent, making it difficult to find a composition that matches its performance, particularly the smooth, clean feeling of dry hair.

Replacing silicone with alternative conditioning agents, such as botanical oils, can be difficult. Botanical oils can negatively impact product viscosity by disrupting the salt-thickening mechanism of the micellar-based surfactant system. Polymeric thickeners (non-associative thickeners) have been used to increase product viscosity; however, when they are used in the amounts needed, dry hair generally looks and/or feels dirty, oily, and/or greasy.

It was found that associative thickeners can be effectively used in relatively low concentrations to achieve suitable product viscosity without dry hair looking and/or feeling dirty, oily, and/or greasy. Associative thickeners rely on non-specific interactions between the end-groups of a thickener molecule and the surrounding formulation (as well as with themselves). The general principle of the thickening mechanism is that, under quiescent conditions, a continuous network is set up throughout the body of the aqueous formulation. In principle, all the components that have a hydrophobic character cooperate in forming a continuous network. Thus, the low molecular weight associative thickeners behave under these conditions as a high molecular weight polymer by association with themselves through their hydrophobes, in a similar way to surfactant micelles or latex particles.

It was found that a shampoo composition that contains a surfactant system comprising an anionic surfactant, a conditioning agent comprising a botanical oil, about 0.5 wt. % to about 3 wt. % of an associative thickener, and a liquid carrier can meet or exceed the performance of shampoo compositions comprising silicone.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All such percentages or weights as they pertain to listed ingredients are based on the active level and, therefore, do not include carriers or by-products that may be included in commercially available materials. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity. All ranges are inclusive and combinable. For example, all numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, 5% or less, or even 3% or less).

“Apply” or “application,” as used in reference to a composition, means to apply or spread the composition onto a human keratinous surface such as the skin or hair.

“Charge density” (“CD”) means the ratio of positive charges on a polymer to the molecular weight of the polymer.

The term “molecular weight” or “Molecular weight” refers to the weight average molecular weight unless otherwise stated. Molecular weight is measured using industry standard method, gel permeation chromatography (“GPC”).

The term “polymer,” as used herein, includes materials whether made by polymerization of one type of monomer or made by two (i.e., copolymers) or more types of monomers.

“Substantially free of” means a composition or ingredient comprises 3 wt. % or less (e.g., 1 wt. % or less, 0.5 wt. % or less, 0.25 wt. % or less) of a subject material. The term “substantially free” as used herein may also mean that the specific material is not added to the composition but may still be present in small quantities in a raw material that is included in the composition.

The term “suitable for application to human hair,” as used herein, means that the compositions or components thereof so described are acceptable for use in contact with human hair and the scalp and skin without undue toxicity, incompatibility, instability, allergic response, and the like.

“Sulfated surfactants” means surfactants that contain a sulfate moiety. Some non-limiting examples of sulfated surfactants are sodium lauryl sulfate, sodium laureth sulfate, ammonium lauryl sulfate, and ammonium laureth sulfate.

The term “water soluble,” as used herein, means that the material is soluble in water in the present composition. In general, the material should be soluble at 25° C. at a concentration of 0.1% by weight of the water solvent, alternatively at 1%, alternatively at 5%, and alternatively at 15%.

### Shampoo Composition

The shampoo compositions herein comprise a surfactant system comprising an anionic surfactant, a conditioning agent comprising a botanical oil; an associative thickener; and a liquid carrier. In some instances, the shampoo composition may include a co-surfactant, for example, to help solubilize the anionic surfactant or another ingredient in the composition. The associative thickener may comprise a hydrophobically modified polyether. The botanical oil may comprise triglycerides and esters of fatty acids, wherein the fatty acids are either saturated, monounsaturated, or polyunsaturated and contain varying chain lengths ranging from C8 to C30. The shampoo composition may also include a cationic conditioning polymer to aid in the appearance and/or feel of hair.

Shampoo compositions herein may have a viscosity of 2,000 mPa-s to 20,000 mPa-s (e.g., 2,500-15,000 mPa-s, 3,000-10,000 mPa-s or 3,500-9,000 mPa-s) according to the Rheology method described in more detail below. It is believed that viscosities in this range are generally preferred by users of liquid shampoo compositions.

### Associative Thickener

The shampoo composition herein includes an associative thickener to build product viscosity without resulting in dry hair looking and/or feeling dirty, oily, and/or greasy. Without being bound by theory, it has been found that the associative thickeners are able to setup a continuous network throughout the body of the aqueous formulation. The hydrophilic body of the associative thickener molecule is fully soluble in the aqueous medium, while the hydrophobic end groups of the associative thickener interact with the hydrophobic core of micelles. The interaction of the associative thickener elongates the micelles to increase the viscosity of micellar system at macroscopic scale.

The composition may include about 0.3 wt. % to about 3 wt. % of an associative thickener. For example, the composition may include an associative thickener in an amount of about 0.4 wt. %, about 0.5 wt. % about 0.6 wt. %, about 0.7 wt. %, about 0.8 wt. %, about 0.9 wt. %, about 1.0 wt. % to about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, or about 3.0 wt. %. It can be important to include enough associative thickener to build up viscosity in the composition. Compositions with insufficient viscosity (i.e., “runny”) may be viewed as poor quality by a consumer. It can also be important to not include too much associative thickener, as it can lead to dry hair looking or feeling dirty, oily, or greasy.

The associative thickener can comprise a hydrophobically modified polyether. The hydrophobically modified polyether can comprise a hydrophobic portion comprising cetyl alcohol, stearyl alcohol, oleyl alcohol, lauryl alcohol or combinations thereof, and a hydrophilic portion of repeating ethylene oxide groups with repeat units from 10-300. The hydrophobically modified polyether can comprise PEG-(60-180)-methylglucose dioleate, PEG-(60-180)-methylglucose trioleate, sorbeth-(60-230)-tetraoleate, PEG-(60-180)-pentaerythrityl tetrastearate, PEG-(60-180)-distearate, PEG-(160-240)-glyceryl stearate, or ceteareth-(60-120)-myristyl glycol.

The associative thickener may comprise PEG-(60-180)-methylglucose dioleate. PEG-(60-180)-methylglucose dioleate is generally described by Formula I illustrated below.

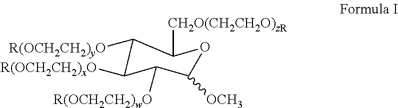

Wherein R is H or C₁₇H₃₃CO (Average 2 R group are C₁₇H₃₃CO) and w+x+y+z=60 to 180.

PEG (60-180) methylglucose dioleate is a molecule composed of a methyl glucose unit with two oleic acid chains attached at the 2 and 6 positions, which are then “capped” with a polyethylene glycol (PEG) chain containing an average of 60-180 ethylene oxide units, essentially creating a hydrophilic backbone with a hydrophobic tail. For example, the associative thickener may comprise PEG 120 methylglucose dioleate.

The associative thickener may comprise PEG-(60-180)-methylglucose trioleate. PEG-(60-180)-methylglucose trioleate is generally described by Formula II illustrated below:

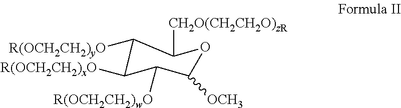

Wherein R is H or C₁₇H₃₃CO (Average 3 R group are C₁₇H₃₃CO) and w+x+y+z=60 to 180.

PEG (60-180) methylglucose trioleate is a molecule composed of a methyl glucose unit with three oleic acid chains, which are then “capped” with a polyethylene glycol (PEG) chain containing an average of 60 to 180 ethylene oxide units, essentially creating a hydrophilic backbone with a hydrophobic tail. For example, the associative thickener may comprise PEG 120 methylglucose trioleate.

The associative thickener may comprise PEG-(60-180)-distearate. distearate is generally described by Formula III illustrated below:

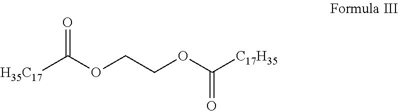

Wherein n=60 to 180.

For example, the associative thickener may comprise PEG-150-distearate.

The associative thickener may comprise Ceteareth-(60-120)-Myristyl Glycol. Ceteareth-(60-120)-Myristyl Glycol is generally described by Formula IV illustrated below:

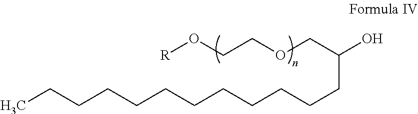

Wherein n=60 to 120 and R-Ceteareth.

The associative thickener may comprise PEG-(160-240)-Glyceryl Stearate. PEG-(160-240)-Glyceryl Stearate is generally described by Formula V illustrated below:

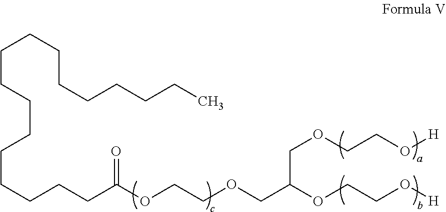

Wherein a+b+c=160 to 240.

For example, the associative thickener may comprise PEG-200-Glyceryl Stearate.

The associative thickener may comprise PEG-(100-200)-pentaerythrityl tetrastearate. PEG-(100-200)-pentaerythrityl tetrastearate is generally described by Formula VI illustrated below:

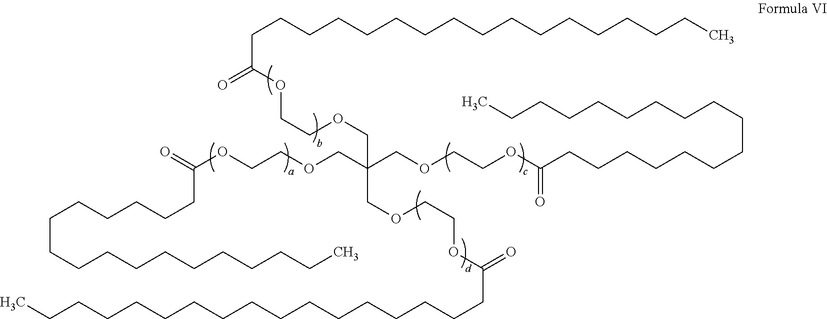

Wherein a+b+c+d=100 to 200.

For example, the associative thickener may comprise PEG-(150)-pentaerythrityl tetrastearate.

The associative thickener may comprise sorbeth-(180-240)-tetraoleate. Sorbeth-(180-240)-tetraoleate is generally described by Formula VII illustrated below:

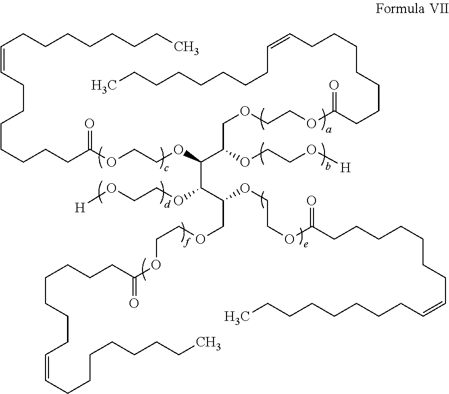

Wherein a+b+c+d+e+f=180 to 240.

For example, the associative thickener may comprise sorbeth-(230)-tetraoleate.

### Botanical Oils

Botanical oils of the type described herein typically are composed of triglycerides and esters of fatty acids. These fatty acids may be either saturated, monounsaturated or polyunsaturated and contain varying chain lengths ranging from C8 to C30. Exemplary fatty acids include saturated fatty acids such as lauric acid (dodecanoic acid), myristic acid (tetradecanoic acid), palmitic acid (hexadecanoic acid), stearic acid (octadecanoic acid), arachidic acid (eicosanoic acid), and lignoceric acid (tetracosanoic acid); unsaturated acids include such fatty acids as palmitoleic (a C16 acid), and oleic acid (a C18 acid); polyunsaturated acids include such fatty acids as linoleic acid (a di-unsaturated C18 acid), linolenic acid (a tri-unsaturated C18 acid), and arachidonic acid (a tetra-unsubstituted C20 acid).

Botanical oils may include but are not limited to, canola oil, safflower oil, argan oil, jojoba oil, coconut oil, shea butter, orange peel wax, tea tree oil, and rice bran oil.

The composition may include 0.2 wt. % to 5.0 wt. % of the botanical oil, alternatively 0.05 wt. % to 2 wt. % botanical oil, alternatively 0.1 wt. % to 1.5 wt. % botanical oil, alternatively 0.15 wt. % to 1 wt. % botanical oil, alternatively 0.2 wt. % to 0.9 wt. % botanical oil, and alternatively 0.2 wt. % to 0.5 wt. % botanical oil of the total composition.

### Anionic Surfactants

The shampoo composition may comprise one or more anionic surfactants to provide cleansing performance. The concentration of the one or more anionic surfactants in the shampoo composition can be sufficient to provide the desired cleaning and lather performance, and generally ranges from 4 wt. % to 25 wt. %, alternatively 8 wt. % to 20 wt. %, alternatively 8 wt. % to 15 wt. %, and alternatively 10 wt. % to 14 wt. %.

Anionic surfactants suitable for use herein include alkyl and alkyl ether sulfates of the formula ROSO₃M and RO(C₂H₄O)ₓSO₃M, wherein R is alkyl or alkenyl of 8 to 18 carbon atoms, x is 1 to 10, and M is a water-soluble cation such as ammonium, sodium, potassium, and triethanolamine cation or salts of the divalent magnesium ion with two anionic surfactant anions. The alkyl ether sulfates may be made as condensation products of ethylene oxide and monohydric alcohols having 8 to 24 carbon atoms. The alcohols can be derived from fats such as coconut oil, palm oil, palm kernel oil, or tallow, or can be synthetic.

Other suitable anionic surfactants include water-soluble salts of the organic, sulfonic acids of the general formula [R¹—SO₃M]. R¹ being a straight chain aliphatic hydrocarbon radical having from 13 to 17 carbon atoms, alternatively from 13 to 15 carbon atoms. M being a water-soluble cation such as ammonium, sodium, potassium, and triethanolamine cation or salts of the divalent magnesium ion with two anionic surfactant anions. These materials are produced by the reaction of SO₂ and O₂ with suitable chain length normal paraffins (C₁₄-C₁₇) and are sold commercially as sodium paraffin sulfonates.

The anionic surfactant may be a combination of sodium lauryl sulfate and sodium laureth sulfate.

Examples of suitable anionic surfactants can include, but are not limited to, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium trideceth sulfate, sodium tridecyl sulfate, sodium methyl lauroyl taurate, sodium methyl cocoyl taurate, sodium lauroyl isethionate, sodium cocoyl isethionate, sodium laurethsulfosuccinate, sodium laurylsulfosuccinate, sodium tridecyl benzene sulfonate, sodium dodecyl benzene sulfonate, or mixtures thereof.

The composition can include one or more sulfate-free anionic surfactants such as sodium, ammonium, or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

Some particularly suitable examples of sulfate-free anionic surfactants can include isethionates, sarcosinates, sulfonates, taurates, or mixtures thereof.

Suitable isethionate surfactants can include the reaction product of fatty acids esterified with isethionic acid and neutralized with sodium hydroxide. Suitable fatty acids for isethionate surfactants can be derived from coconut oil or palm kernel oil including amides of methyl tauride. Non-limiting examples of isethionates can be selected from the group consisting of sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, or mixtures thereof.

An amino acid-based anionic surfactant can be a sarcosinate, for instance an acyl sarcosinate. Non-limiting examples of sarcosinates can be selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroylglutamate/lauroylsarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium oleoyl sarcosinate, sodium palmitoyl sarcosinate, TEA-cocoyl sarcosinate, TEA-lauroyl sarcosinate, TEA-oleoyl sarcosinate, TEA-palm kernel sarcosinate, and combinations thereof.

Non-limiting examples of sulfonates can include alpha olefin sulfonates, alkyl olefin sulfonates, linear alkylbenzene sulfonates, sodium laurylglucosides hydroxypropylsulfonate, and combinations thereof.

### Co-Surfactants

The shampoo composition may include a co-surfactant selected from anionic surfactants, amphoteric surfactants, zwitterionic surfactants, non-ionic surfactants, and combinations of these. Some non-limiting examples of anionic surfactants include non-taurate, non-sulfate anionic surfactants such as isethionates, carboxylates, sulfonates (e.g., alpha olefin sulfonates, alkyl olefin sulfonates, linear alkylbenzene sulfonates, alkyl glyceryl sulfonates, sodium laurylglucosides hydroxypropylsulfonate), branched alkyl sulfates, sulfosuccinates, sulfoacetates, sulfolaurates, amino acid-based surfactants (e.g., glycinates, sarcosinates, alaninates, glutamates), lactate- and lactylate-based surfactants (e.g., sodium lauroyl lactate and sodium lauroyl lactalyte), phosphate ester surfactants and combinations thereof.

Some non-limiting examples of amphoteric and/or zwitterionic surfactants include derivatives of aliphatic secondary and tertiary amines in which one of the aliphatic substituents contains from 8 to 18 carbon atoms and one aliphatic substituent contains an anionic group such as a carboxy, sulfonate, phosphate, or phosphonate group. Zwitterionic surfactants are surfactants whose polar functional group has two permanent charges that do not change with changing pH. Amphoteric surfactants have polar functional groups whose charge depends on the pH of the solution and can exhibit different charges as the pH changes from acid to neutral to basic, ranging from cationic to zwitterionic and potentially even to anionic. Some non-limiting examples of zwitterionic surfactants include amidosulfobetaines, amidopropylbetaines, hydroxysultaines, amidopropyl hydroxysultaines, lauryl hydroxysultaine, coco-hydroxysultaine, and combinations thereof. Some non-limiting examples of amphoteric surfactants include amphoacetates, amphodiacetates, betaines (including alkyl betaines, coco-betaine, and lauryl betaine), amidobetaines (e.g., cocamidopropyl betaine and lauramidopropyl betaine), propionates, hydroxysultaines, and combinations thereof.

Some non-limiting examples of non-ionic surfactants include glyceryl esters of alkanoic acids, polyglyceryl esters of alkanoic acids, propylene glycol esters of alkanoic acids, sorbitol esters of alkanoic acids, alkanolamides, alkoxylated amides, alkyl glycosides, alkyl polyglucosides, decyl glucoside, lauryl glucoside, coco-glucoside, acyl glucamides, amine oxides and combinations thereof. Some particularly suitable examples of non-ionic surfactants include cocamide, cocamide MEA, cocamide methyl MEA (CMMEA), PPG-2 cocamide, PPG-2 hydroxyethyl cocamide, cocamide diethanolamine (DEA), PPG-2 hydroxyethyl isostearamide, lauroyl/myristoyl methyl glucamide, capryloyl/caproyl methyl glucamide, cocoyl methyl glucamide, decyl glucoside, coco-glucoside, lauryl glucoside, lauramine oxide, cocamine oxide and combinations thereof.

More specific examples of the optional co-surfactants described above are disclosed in US 2019/0105246, US 2018/0098923, U.S. Pat. No. 9,271,908, WO 2020/016097, and Mccutcheon's Emulsifiers and Detergents, 2019, MC Publishing Co.

The co-surfactant may be present in the shampoo compositions at 1 wt. % to 15 wt. % (e.g., 2 to 10 wt. %, 3 to 9 wt. %, 4 to 8 wt. %, or even 5 to 7 wt. %). The amount of co-surfactant in the composition can be important and should be tailored to balance solubility and/or viscosity building with cleaning and/or conditioning benefits. For example, too much amphoteric co-surfactant can make the surfactant system less salt-tolerant and may impede the ability of the surfactant system to form a suitable coacervate upon dilution with water.

### Cationic Polymer

The shampoo compositions herein may include a cationic polymer. The compositions may include 0.05 wt. % to 3 wt. % of a cationic polymer (e.g., 0.1-2 wt. %, or even 0.2-0.8 wt. %) to provide an improved appearance, feel, or deposition benefits to hair or skin. The cationic polymer can have a weight average molecular weight of 50 kDa to 5 MDa (e.g., 500 kDa-4 MDa, 1-3 MDa, 1.2-2 MDa, or even 1.4-1.8 MDa) and a charge density of 0.2 meq/g to 12 meq/g (e.g., 0.4-10 meq/g, 0.4-5 meq/g, 0.4-4 meq/g, 0.4-3 meq/g, or even 0.4-2 meq/g). The charge densities can be measured at the pH of the intended use of the shampoo composition, which can be pH 3 to pH 9 (e.g., pH 4-8 or pH 4.5-6.5).

The cationic polymers may include cationic, nitrogen-containing moieties such as quaternary ammonium or cationic protonated amino moieties. The cationic protonated amines can be primary, secondary, or tertiary amines, depending upon the particular species and the selected pH of the composition. Anionic counterions can be used in association with the cationic polymers, as long as the polymers remain soluble. Examples of suitable counterions include halide counterions (e.g., chloride, fluoride, bromide, iodide).

Some non-limiting examples of cationic polymers include copolymers of vinyl monomers having cationic protonated amine or quaternary ammonium functionalities with water-soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone. Some non-limiting examples of cationic protonated amino and quaternary ammonium monomers include vinyl compounds substituted dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Additional nonlimiting examples of cationic polymers include copolymers of 1-vinyl-2-pyrrolidone and 1-vinyl-3-methylimidazolium salt (e.g., chloride salt) (referred to in the industry by the Shampoo Products Council (“PCPC”) as Polyquaternium-16); copolymers of 1-vinyl-2-pyrrolidone and dimethylaminoethyl methacrylate (Polyquaternium-11); cationic diallyl quaternary ammonium-containing polymers, including, for example, dimethyldiallylammonium chloride homopolymer, copolymers of acrylamide and dimethyldiallylammonium chloride (Polyquaternium-6 and Polyquaternium-7, respectively); amphoteric copolymers of acrylic acid including copolymers of acrylic acid and dimethyldiallylammonium chloride (Polyquaternium-22), terpolymers of acrylic acid with dimethyldiallylammonium chloride and acrylamide (Polyquaternium-39), and terpolymers of acrylic acid with methacrylamidopropyl trimethylammonium chloride and methylacrylate (Polyquaternium-47). In some aspects, suitable cationic substituted monomers include cationic substituted dialkylaminoalkyl acrylamides, dialkylaminoalkyl methacrylamides, and combinations thereof. The cationic polymer can be AM:TRIQUAT which is a copolymer of acrylamide and 1,3-Propanediaminium, N-[2-[[[dimethyl[3-[(2-methyl-1-oxo-2-propenyl)amino]propyl]ammonio]acetyl]amino]ethyl]2-hydroxy-N,N,N′,N′,N′-pentamethyl-, trichloride (Polyquaternium-76). AM:TRIQUAT may have a charge density of 1.6 meq/g and a molecular weight of 1.1 MDa.

The cationic monomer may be polymethyacrylamidopropyl trimonium chloride, available under the trade name Polycare® 133, from Solvay (Brussels, Belgium). Copolymers of the cationic monomer may also be suitable, and the charge density of the total copolymer can be 2.0 meq/g to 4.5 meq/g.

Other cationic polymers include polysaccharide polymers, such as cationic cellulose derivatives and cationic starch derivatives. In certain embodiments, a cationic cellulose polymer can be selected from the salts of hydroxyethyl cellulose reacted with trimethyl ammonium substituted epoxide, referred to in the industry (PCPC) as Polyquaternium-10 and available from Dow Chemical Company as UCARE™ JR-30M, KG-30 M and LR-30M. Other examples of cationic cellulose polymers include polymeric quaternary ammonium salts of hydroxyethyl cellulose reacted with lauryl dimethyl ammonium-substituted epoxide referred to in the industry (PCPC) as Polyquaternium-24.

Further examples of cationic polymers include cationic guar gum derivatives, such as guar hydroxypropyltrimonium chloride, such as the Jaguar® series available from Solvay and the N-Hance™ and AquaCat™ series from Ashland (Wilmington, Delaware). Additional disclosure of cationic guar gum derivatives can be found in U.S. Pat. No. 6,930,078.

The cationic polymer may include a synthetic cationic polymer or derivative thereof present at 0.025 wt. % to about 5 wt. %. The synthetic cationic polymers may be generally water-soluble or dispersible and non-crosslinked. The synthetic cationic polymer may be a copolymer that includes one or more cationic monomer units and one or more nonionic or anionic monomer units, as long as the copolymer has a net positive charge. Synthetic cationic polymers can have a cationic charge density of 0.5 meq/g to 12 meg/g and an average molecular weight of 1 kDa to 5 MDa. Some non-limiting examples of synthetic cationic polymers are described in US 2003/0223951.

### Liquid Carrier

The shampoo composition may include an aqueous carrier. The shampoo composition may include 20 wt. % to 95 wt. % of an aqueous carrier such as water and/or a water-miscible solvent. The type and amount of aqueous carrier should be selected to provide the composition with the desired rheological properties. The liquid carrier can be water with, e.g., less than 10 wt. %, 7 wt. %, 5 wt. %, 3 wt. %, 1 wt. %, 0.5 wt. % or even 0 wt. % miscible organic solvent. Some nonlimiting examples of organic solvents include lower alkyl alcohols (e.g., ethanol and isopropanol) and polyhydric alcohols (e.g., propylene glycol, hexylene glycol, glycerin, and propane diol). A composition can include ≥50 wt. %, alternatively ≥60 wt. %, alternatively ≥70 wt. %, alternatively ≥75 wt. %, of a liquid carrier.

### Optional Ingredients

The shampoo compositions described herein may include a variety of optional ingredients to tailor the properties and characteristics of the composition, as desired. The optional ingredients may be materials that are commonly included in compositions of the type. The optional ingredients should be physically and chemically compatible with the essential components of the shampoo composition and should not otherwise unduly impair the stability, aesthetics, or performance of the composition. Individual concentrations of optional components can generally range from 0.001 wt. % to 10 wt. %.

Some non-limiting examples of optional ingredients that can be included in the shampoo compositions herein include deposition aids, cationic polymers, conditioning agents (including gel network, triglyceride oils, hydrocarbon oils, fatty esters, silicones), anti-dandruff agents (e.g., zinc pyrithione, zinc carbonate, piroctone olamine, piroctone, ciclopirox, rilopirox, MEA-Hydroxyoctyloxypyridinone, azoxystrobin, sulfur, azoles, salicylic acid and selenium sulfide, 1,10-phenanthroline), anti-microbial agents, suspending agents, viscosity modifiers, dyes, pigments, nonvolatile solvents or diluents (water soluble and insoluble), pearlescent aids, foam boosters, pediculocides, pH adjusting agents, perfumes, preservatives, chelants, proteins, vitamins, amino acids, skin active agents, sunscreens, UV absorbers, stabilizers, and combinations of these.

### Dispersed Gel Network

The shampoo composition may include a dispersed gel network phase to provide a suitable cleaning benefit to the composition in combination with the anionic surfactant. The shampoo composition can contain 1 wt. % to 12 wt. % dispersed gel network phase, alternatively 1 wt. % to 7 wt. % dispersed gel network phase, alternatively 1.25 wt. % to 6 wt. %, alternatively 1.5 wt. % to 5 wt. %, and alternatively 2 wt. % to 4 wt. %.

Suitable dispersed gel networks can be formed by combining a fatty alcohol and a gel network surfactant in a suitable ratio and heating the dispersion to a temperature above the melting point of the fatty alcohol. Additional details of suitable gel networks are described in G. M. Eccleston, “Functions of Mixed Emulsifiers and Emulsifying Waxes in Dermatological Lotions and Creams”, Colloids and Surfaces A: Physiochem, and Eng. Aspects 123-124 (1997) 169-182; and by G. M Eccleston, “The Microstructure of Semisolid Creams”, Pharmacy International, Vol. 7, 63-70 (1986), each of which is incorporated by reference herein.

It may be desirable to pre-form the gel network phase, which means that at least fifty percent of the mixture of the fatty alcohol, gel network surfactant, and liquid carrier are in a substantially solid crystalline phase prior to addition to the other components of the shampoo composition. When the dispersed gel network is pre-formed, the gel network component can be prepared as a separate pre-mix, which, after being cooled, can be subsequently incorporated with an anionic surfactant and any other components of a shampoo composition. While not intending to be limited by theory, it is believed that incorporation of a pre-formed gel network component with the anionic surfactant and other components of the shampoo composition allows the formation of a substantially equilibrated lamellar dispersion (“ELD”) in the final composition.

The ELD is a dispersed lamellar or vesicular phase resulting from the pre-formed gel network component substantially equilibrating with the anionic surfactants, carrier, and other optional components of a shampoo composition. This equilibration occurs upon incorporation of the pre-formed gel network component with the other components of a shampoo composition and can be effectively completed within 24 hours after incorporation.

### Gel Network Fatty Alcohol

The dispersed gel network phase may include a fatty alcohol (e.g., C10-C40 fatty alcohols) at 0.05 wt. % or more of the composition (e.g., 0.05 wt. % to 25 wt. %, 0.5 wt. % to 20 wt. %, or 1 wt. % to 8 wt. %). The fatty alcohol may be a straight or branched chain and can be saturated or unsaturated. As can be appreciated, suitable fatty alcohols can be of natural, vegetable, or synthetic origin. In some aspects, it may be desirable to mix several fatty alcohols to provide a dispersed gel network phase with a melt transition temperature of 38° C. or greater such as, for example, a mixture of cetyl alcohol and stearyl alcohol at a ratio of between 20:80 and 80:20. Some non-limiting examples of fatty alcohols that may be suitable for use herein include cetyl alcohol, stearyl alcohol, arachidyl alcohol, behenyl alcohol, C₂₁ fatty alcohol (1-heneicosanol), C₂₃ fatty alcohol (1-tricosanol), C₂₄ fatty alcohol (lignoceryl alcohol, 1-tetracosanol), C₂₆ fatty alcohol (1-hexacosanol), C₂₈ fatty alcohol (1-octacosanol), C₃₀ fatty alcohol (1-triacontanol), C₂₀₋₄₀ alcohols (e.g., Performacol® 350 and 425 Alcohols, available from New Phase Technologies), C₃₀₋₅₀ alcohols (e.g., Performacol® 550 Alcohol), C₄₀₋₆₀ alcohols (e.g., Performacol® 700 Alcohol), or mixtures thereof.

The shampoo compositions may include fatty alcohol and at least a portion, if not most of the fatty alcohol is part of the dispersed gel network phase. The shampoo composition can include a fatty acid in an amount of least 1 wt. %, alternatively 1 wt. % to 8 wt. %, alternatively 1.25 wt. % to 6 wt. %, alternatively 1.5 wt. % to 5 wt. %, alternatively 2 wt. % to 5 wt. %, and alternatively 2 wt. % to 4 wt. %, of the shampoo composition.

The weight ratio of the fatty alcohol to the gel network surfactant in the gel network component may be greater than 1:9, alternatively 1:5 to 100:1, and alternatively 1:1 to 50:1.

### Gel Network Surfactant

The shampoo compositions may include a gel network surfactant. The composition may include a gel network surfactant at 0.01 wt. % to 15 wt. %, alternatively 0.1 wt. % to 10 wt. %, and alternatively 0.2 wt. % to 5 wt. %, of the shampoo composition.

The gel network surfactant can be any suitable anionic, zwitterionic, amphoteric, cationic, nonionic surfactant, or mixtures thereof, as described herein. In some examples, the gel network surfactant is free of or substantially free of sulfates. The anionic surfactant and the gel network surfactant can be independently selected and can be the same or different. In some aspects, the gel network surfactant has a hydrophobic tail group with a chain length of 10 to 40 carbon atoms. The hydrophobic tail group may be alkyl, alkenyl (containing up to 3 double bonds), alkyl aromatic, or branched alkyl. Mixtures of more than one gel network surfactant can also be used. Some non-limiting examples of gel network surfactants are disclosed in US 2006/0024256.

### Gel Network Water or Suitable Solvent

The dispersed gel network phase may also include water or suitable solvents. The water or suitable solvent and the gel network surfactant together contribute to the swelling of the fatty alcohol. This, in turn, leads to the formation and stability of the gel network. As used herein, the term “suitable solvent” refers to any solvent that can be used in place of or in combination with water in the formation of the gel network.

The shampoo compositions may comprise water or suitable solvents as part of the pre-formed dispersed gel network component in an amount suitable to achieve a gel network when combined with fatty alcohol and gel network surfactant according to the present invention. The gel network water or suitable solvent may be present in an amount of at least 0.05 wt. %, of the total shampoo composition.

### Method of Making a Shampoo Composition

The shampoo compositions described herein are generally prepared by conventional methods such as those known in the art of making the compositions. Such methods typically involve mixing the ingredients in one or more steps to a relatively uniform state, with or without heating, cooling, application of vacuum, and the like. The compositions are prepared to optimize stability (physical stability, chemical stability, photostability) and/or delivery of the active materials.

### Method of Use

The shampoo compositions described herein can be used in a conventional manner for cleansing and conditioning of hair. Effective amounts of the composition for use generally range from 1 g to 50 g (e.g., 1 g to about 20 g). Generally, a method of treating hair can include applying the shampoo composition to the hair. For example, an effective amount of the shampoo composition can be applied to the hair, that has been wetted with water, and then the composition can be rinsed off. Application to the hair typically includes working the composition through the hair such that most or all the hair is contacted with the composition. The shampoo composition can be used as a liquid. The method for treating the hair or skin can include the steps of (a) wetting the hair or skin with water; (b) applying an effective amount of the shampoo composition to the hair, and (c) rinsing the applied areas of hair with water. These steps can be repeated as many times as desired to achieve the desired cleansing and conditioning benefit.

### TEST METHODS

### Cone/Plate Viscosity Measurement

The viscosities of the examples are measured by a Cone/Plate Controlled Stress Brookfield Rheometer R/S Plus, by Brookfield Engineering Laboratories, Stoughton, MA. The cone used (Spindle C-75-1) has a diameter of 75 mm and 1° angle. The liquid viscosity is determined using a steady-state flow experiment at a constant shear rate of 2 s-1 and a temperature of 27° C. The sample size is 2.5 ml to 3 ml and the total measurement reading time is 3 minutes. For viscosity measurements after aging for one week, the formulations were kept at 40° C. for the given time. After the aging period, the sample was allowed to cool down to room temperature (i.e., 20-25° C.) before running the viscosity measurement according to the method described above.

### Hair Texture Analysis Method

### Hair Substrate

Round ponytail hair tresses of general population, Chinese hair measuring 4 grams in weight and 8 inches in length were purchased from International Hair Importers & Products Inc., 87-29 Myrtle Ave., Glendale, NY 11385 and used in the hair treatment procedure that follows. Each test product was applied to 3 separate hair tresses (n=3).

### Hair Treatment

For each wash cycle, 0.4 grams (0.1 g/gram hair) of a shampoo (e.g., Composition A) was applied to a hair tress that was first prewetted with 100-105° F. water and then squeegeed to remove any excess water. The product was applied down the hair tress in a zig-zag pattern, equally to the front and back of the hair tress (0.2 grams per side). The hair tress was then brushed, using a Goody Brush with large, stiff, plastic bristles, alternating down the front and back for 30 strokes at approximately 1 stroke per second for a total of 30 seconds. The tress was then rinsed using 100-105° F. water for 30 seconds while milking the switch at approximately 1 stroke per second. The tress was squeezed to remove any excess water. To complete a second wash cycle, the test product was then reapplied to the hair tress a second time following the same procedure described above and then rinsed from the hair tress. Multiple wash cycles can be accomplished by repeating the same procedures described above. For example, to assess the hair tress after 5 treatment cycles (“Cycle-5”), the wash cycle described above was completed five times before the measurement was conducted.

### Measurement of the Detangling Force for Combing Force

The force required to comb a hair tress after treatment (wet and dry) was measured using the Texture Analyzer TA-XT Plus (manufactured by Stable Micro Systems), Instron 5542 (manufactured by Instron) or equivalent force measurement device. This method is an industry standard method for measuring wet/dry hair combing forces, disclosed by TRI Princeton.

The hair tress is placed within the holder of the Texture Analyzer, fixed at the root end of the hair tress. The hair tress is positioned within the combs in series and then pulled through the combs by the Texture Analyzer, while the average force to pull the tress through each comb is recorded (=1 combing stroke). The hair tress is disengaged from the combs and returned to its pre-combing position. The hair tress is then combed 9 more times using the same combing procedure. The measured values represent average gram force (gf) values for combing strokes 2 through 6 of a 10-stroke test. Lower values represent less combing force and higher conditioning, which is desired.

### EXAMPLES

The following data and examples are provided to help illustrate the shampoo compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified. The levels given reflect the weight percent of the active material, unless otherwise specified.

### Example 1: Viscosity Evaluation

Shampoos that use botanical oils as conditioning agents often lose viscosity, becoming too watery for consumer preference. Traditional thickeners have proven ineffective at boosting viscosity without negatively affecting other consumer-desired properties.

In this example, in exemplary embodiments of shampoo compositions, coconut oil was used as a botanical oil conditioning agent and PEG-120-methylglucose dioleate was used as an associative thickener in amounts varying from 0 to 2 wt % relative to the total shampoo weight. This example demonstrates the viscosity benefit of adding PEG-120-methylglucose dioleate to shampoo compositions comprising botanical oils. Shampoo viscosity ranging from about 2000 mPa-s to about 20,000 mPa-s is suitable for consumer use.

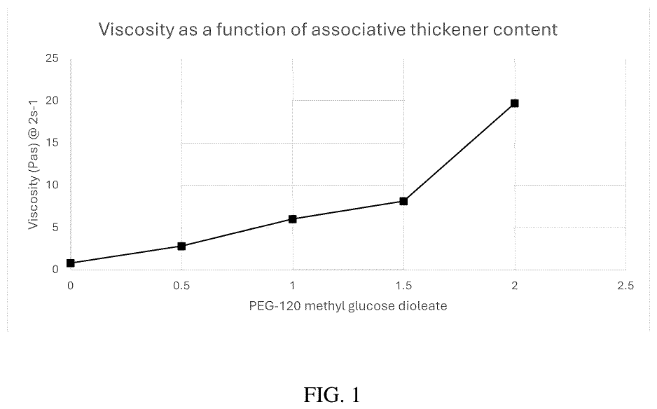

Table 1 provides composition and viscosity data for Relative Example R1, Examples 1~4 and Comparative Example C1. Coconut oil and PEG-120-methylglucose dioleate were not present in C1. Viscosity was measured according to the methods described herein. The viscosity results are illustrated in FIG. 1.

TABLE 1

| | R1 | Ex. 1 | Ex. 2 | Ex. 3 | Ex. 4 | C1 |
|---|---|---|---|---|---|---|
| Sodium Laureth Sulfate | 11.6 | 11.6 | 11.6 | 11.6 | 11.6 | 11.6 |
| Sodium Lauryl Sulfate | 1.4 | 1.4 | 1.4 | 1.4 | 1.4 | 1.4 |
| Cocamidopropyl Betaine | 2 | 2 | 2 | 2 | 2 | 2 |
| Coconut oil | 2 | 2 | 2 | 2 | 2 | 0 |
| PEG-120-methylglucose | 0 | 0.5 | 1 | 1.5 | 2 | 0 |
| dioleate ⁽¹⁾ | | | | | | |
| Guar Hydroxypropyl | 0.4 | 0.4 | 0.4 | 0.4 | 0.4 | 0.4 |
| Trimonium Chloride | | | | | | |
| Gel Network Premix from | 2.3 | 2.3 | 2.3 | 2.3 | 2.3 | 2.3 |
| Table 2 | | | | | | |
| Hydrogenated Castor Oil | 0.06 | 0.06 | 0.06 | 0.06 | 0.06 | 0.06 |
| Citric Acid / Sodium Citrate | Adjust to pH ~5-7 | | | | | |
| Dihydrate / HCl / NaOH | | | | | | |
| Water, Fragrance, | Q.S. to 100 | | | | | |
| Preservatives | | | | | | |
| Viscosity (Pa-s, after 1 week | 0.8 | 2.8 | 6 | 8.1 | 19.7 | 9.1 |
| at 40° C.) | | | | | | |

⁽¹⁾ Antil ® 127 from Evonik

TABLE 2

| | Ex. 5 | Ex.6 | Ex. 7 | Ex. 8 | Ex. 9 |
|---|---|---|---|---|---|
| Sodium Laureth Sulfate | 11.6 | 11.6 | 11.6 | 11.6 | 11.6 |
| Sodium Lauryl Sulfate | 1.4 | 1.4 | 1.4 | 1.4 | 1.4 |
| Cocamidopropyl Betaine | 2 | 0 | 2 | 0 | 0 |
| Coconut oil | 2 | 2 | 2 | 2 | 2 |
| PEG-120-methylglucose triioleate ⁽²⁾ | 1 | 2 | | | |
| PEG-150 distearate ⁽³⁾ | | | 1 | 2 | |
| PEG-200 Glyceryl Stearate ⁽⁴⁾ | | | | | 1 |
| PEG-150 pentaerythrityl tetrastearate ⁽⁵⁾ | | | | | |
| Sorbeth-230 tetraoleate ⁽⁶⁾ | | | | | |
| Guar Hydroxypropyl Trimonium Chloride | 0.4 | 0.4 | 0.4 | 0.4 | 0.4 |
| Gel Network Premix | 2.3 | 2.3 | 2.3 | 2.3 | 2.3 |
| Hydrogenated Castor Oil | 0.06 | 0.06 | 0.06 | 0.06 | 0.06 |
| Dimethionol, Dimethicone ⁽²⁾ | | | | 2 | 2 |
| Citric Acid/Sodium Citrate Dihydrate/ | Adjust to pH ~5-7 | | | | |
| HCl/NaOH | | | | | |
| Water, Fragrance, Preservatives | Q.S. to 100 | | | | |
| Viscosity | 8.1 | 18.9 | 5.3 | 13.5 | 6.5 |

TABLE 3

| | Ex. 10 | Ex. 11 | Ex. 12 | Ex. 13 | Ex. 14 |
|---|---|---|---|---|---|
| Sodium Laureth Sulfate | 11.6 | 11.6 | 11.6 | 11.6 | 11.6 |
| Sodium Lauryl Sulfate | 1.4 | 1.4 | 1.4 | 1.4 | 1.4 |
| Cocamidopropyl Betaine | 0 | 0 | 0 | 0 | 0 |
| Coconut oil | 2 | 2 | 2 | 2 | 2 |
| PEG-120-methylglucose triioleate ⁽²⁾ | | | | | |
| PEG-150 distearate ⁽³⁾ | | | | | |
| PEG-200 Glyceryl Stearate ⁽⁴⁾ | 2 | | | | |
| PEG-150 pentaerythrityl tetrastearate ⁽⁵⁾ | | 1 | 2 | | |
| Sorbeth-230 tetraoleate ⁽⁶⁾ | | | | 1 | 2 |
| Guar Hydroxypropyl Trimonium Chloride | 0.4 | 0.4 | 0.4 | 0.4 | 0.4 |
| Gel Network Premix | 2.3 | 2.3 | 2.3 | 2.3 | 2.3 |
| Hydrogenated Castor Oil | 0.06 | 0.06 | 0.06 | 0.06 | 0.06 |
| Dimethionol, Dimethicone ⁽⁷⁾ | 2 | 2 | 2 | 2 | 2 |
| Citric Acid/Sodium Citrate Dihydrate/ | Adjust to pH ~5-7 | | | | |
| HCl/NaOH | | | | | |
| Water, Fragrance, Preservatives | Q.S. to 100 | | | | |
| Viscosity | 22.3 | 10.3 | 20.8 | 9.5 | 23.6 |

⁽²⁾ Glucamate LT
⁽³⁾ Rewopal PEG 6000 DS
⁽⁴⁾ Antil 500
⁽⁵⁾ Crothix Liquid -LQ
⁽⁶⁾ Sorbithix L-100
⁽⁷⁾ Belsil ® DM5500 from Wacker Chemie AG

Table 4 shows the composition of the gel network premix in the Examples provided herein.

TABLE 4

| | (wt. %) |
|---|---|
| Sodium Laureth-1 Sulfate | 11.00 |
| Stearyl Alcohol | 7.71 |
| Cetyl Alcohol | 4.29 |
| Water | QS |

FIG. 1 illustrates the viscosity of shampoo compositions for Relative Example R1 and Examples 1-4. This result shows that the compositions of Example 1-4 had suitable viscosities, which emphasizes that PEG-120-methylglucose dioleate can be used in combination with botanical oils to provide shampoo compositions having consumer-acceptable viscosities.

### Example 2. Conditioning and Detangling Evaluation

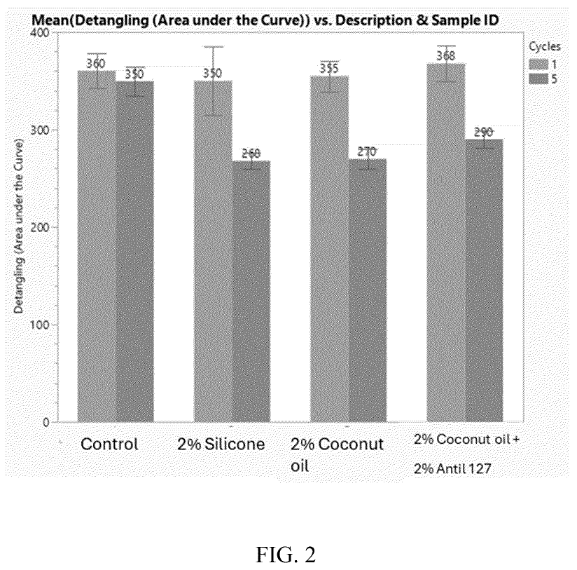

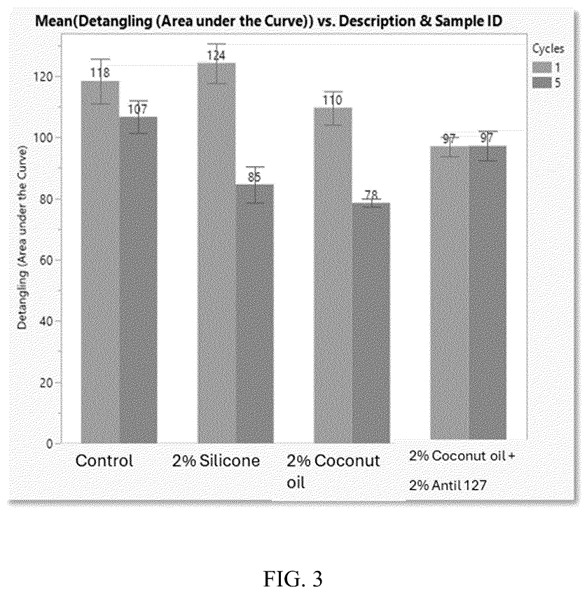

The conditioning and detangling effect of four compositions was evaluated. Table 5 provides composition and detangling force data for Example 4 and Comparative Examples C2, C3, and C4. The detangling force was measured according to the methods described herein. The detangling force results are illustrated in FIG. 2 and FIG. 3. FIG. 2 shows results for wet hair and FIG. 3 shows results for dry hair.

TABLE 5

| | Ex. 4 | C2 | C3 | C4 |
|---|---|---|---|---|
| Sodium Laureth Sulfate | 11.6 | 11.6 | 11.6 | 11.6 |
| Sodium Lauryl Sulfate | 1.4 | 1.4 | 1.4 | 1.4 |
| Cocamidopropyl Betaine | 2 | 0 | 2 | 0 |
| Coconut oil | 2 | 0 | 2 | 0 |
| PEG-120-methylglucose dioleate ⁽⁸⁾ | 2 | 0 | 0 | 0 |
| Guar Hydroxypropyl Trimonium | 0.4 | 0.4 | 0.4 | 0.4 |
| Chloride | | | | |
| Gel Network Premix from Table 2 | 2.3 | 2.3 | 2.3 | 2.3 |
| Hydrogenated Castor Oil | 0.06 | 0.06 | 0.06 | 0.06 |
| Dimethiconol, Dimethicone⁽⁹⁾ | | | | 2 |

| | |
|---|---|
| Citric Acid/Sodium Citrate | Adjust to pH ~5-7 |
| Dihydrate/HCl/NaOH | |
| Water, Fragrance, Preservatives | Q.S. to 100 |

Detangling force for combing force, gf *

| | | | | |
|---|---|---|---|---|
| Wet-Work for detangling (Average)- | 368 | 360 | 355 | 350 |
| Cycle-1 | | | | |
| Wet-Work for detangling (Std | 26.67 | 33.4 | 30.28 | 37.8 |
| Error)-Cycle-1 | | | | |
| Wet-Work for detangling (Average)- | 290 | 350 | 270 | 260 |
| Cycle-5 | | | | |
| Wet-Work for detangling (Std | 18.96 | 25.95 | 17.35 | 17.84 |
| Error)-Cycle-5 | | | | |
| Dry-Work for detangling (Average)- | 97 | 118 | 110 | 124 |
| Cycle-1 | | | | |
| Dry-Work for detangling (Std | 8.7 | 8.5 | 9.5 | 10.07 |
| Error)-Cycle-1 | | | | |
| Dry-Work for detangling (Average)- | 97 | 107 | 78 | 85 |
| Cycle-5 | | | | |
| Dry-Work for detangling (Std | 8.5 | 8.23 | 6.8 | 6.16 |
| Error)-Cycle-5 | | | | |

*Values represent average (mean) gram force (gf) values for combing strokes 2 thru 6 (of a 10-stroke test), using a sample size of n = 3 hair tresses per product. Lower values represent less combing force and higher conditioning, which is preferred for the shampoo composition herein. The composition for the gel network premix is provided in Table 2.
⁽⁸⁾ Antil ® 127 from Evonik
⁽⁹⁾Belsil ® DM5500 from Wacker Chemie AG

As can be seen in FIG. 2, for wet hair, the detangling results showed that Example 5 (2 wt. % coconut oil+2 wt. % PEG-120-methylglucose dioleate) performed similarly to C3 (2 wt. % coconut oil) and C4 (2 wt. % silicone). This result is beneficial because C4 is the composition comprising silicone. Thus, the composition comprising botanical oil and PEG-120-methylglucose dioleate performed comparably to a composition comprising silicone for wet detangling testing. Thus, from a consumer perspective, the composition of Example 5 and the composition of C4 perform consistently for wet detangling. Moreover, because Example 5 comprises both botanical oil and PEG-120-methylglucose dioleate, the viscosity of the composition of Example 5 will be acceptable for consumer use as well. In contrast, the composition of C3 comprises coconut oil for conditioning but does not contain PEG-120-methylglucose dioleate for thickening. Thus, the viscosity for C3 will be too runny for consumer satisfaction.

The results for dry detangling testing (FIG. 3) essentially mirror those for wet detangling testing. That is, the detangling force for the composition of Example 5 (2 wt. % coconut oil+2 wt. % PEG-120-methylglucose dioleate) was similar to that for compositions C3 (2 wt. % coconut oil) and C4 (2 wt. % silicone). Thus, the composition comprising botanical oil and PEG-120-methylglucose dioleate performed comparably to a composition comprising silicone for dry detangling testing. Accordingly, from a consumer perspective, the composition of Example 5 and the composition of C4 perform consistently for dry detangling. Moreover, because Example 5 comprises both botanical oil and PEG-120-methylglucose dioleate, the viscosity of the composition of Example 5 will be acceptable for consumer use as well. In contrast, the composition of C3 comprises coconut oil for conditioning but does not contain PEG-120-methylglucose dioleate for thickening. Thus, the viscosity for C3 will be too runny for consumer satisfaction.

### Example 3. Viscosity Evaluation for Sulfate-Free Surfactant Systems

In this example, the viscosity of exemplary embodiments of shampoo compositions containing sulfate-free surfactant systems was evaluated. Coconut oil was used as a botanical oil conditioning agent and PEG-120-methylglucose dioleate was used as an associative thickener in amounts varying from 0 to 1 wt % relative to the total shampoo weight. This example demonstrates the viscosity benefit of adding PEG-120-methylglucose dioleate to shampoo compositions comprising botanical oils and sulfate-free surfactant systems.

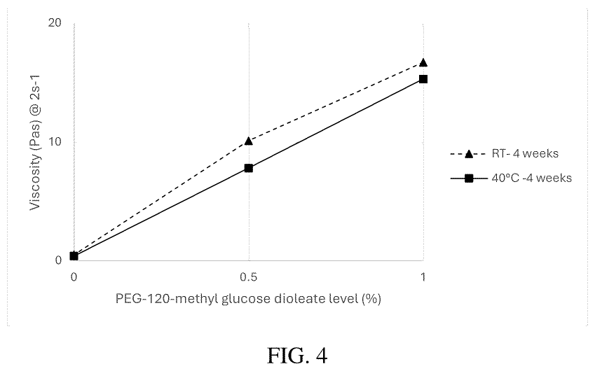

Table 6 provides composition and viscosity data for Example 15, Example 16, and Comparative Example C5. Viscosity was measured according to the methods described herein. The viscosity was measured after 4 weeks at 25° C. and after 4 weeks at 40° C. The viscosity results are illustrated in FIG. 4.

TABLE 6

| | Ex. 15 | Ex. 16 | C5 |
|---|---|---|---|
| Alkyl Olefin Sulfonate ⁽¹⁰⁾ | 12 | 12 | 12 |
| Cocamidopropyl Betaine | 4 | 4 | 4 |
| Cocamide MEA ⁽¹¹⁾ | 2 | 2 | 2 |
| Coconut oil | 1 | 1 | 1 |
| PEG-120-methylglucose dioleate ⁽¹²⁾ | 1 | 0.5 | 0 |
| Guar Hydroxypropyl Trimonium | 0.4 | 0.4 | 0.4 |
| Chloride ⁽¹³⁾ | | | |
| Hydrogenated Castor Oil ⁽¹⁴⁾ | 0.06 | 0.06 | 0.06 |
| Citric Acid / Sodium Citrate Dihydrate/ | Adjust to pH ~5-7 | | |
| HCl/NaOH | | | |
| Water, Fragrance, Preservatives | Q.S. to 100 | | |
| Viscosity (Pas- after 4 weeks @25° C.) | 16.7 | 10.1 | 0.5 |
| Viscosity (Pas- after 4 weeks @40° C.) | 15.3 | 7.8 | 0.4 |

⁽¹⁰⁾ Calsoft ® AOS-40 (Pilot Chemical)
⁽¹¹⁾ Comperlan ® CMEA (BASF)
⁽¹²⁾ Antil 127 from Evonik
⁽¹³⁾ N-Hance 3196 from Ashland
⁽¹⁴⁾ Thixcin ® R from Elementis

Each composition contained 1 wt. % coconut oil, with the amount of PEG-120-methylglucose dioleate varying from 0 wt. % to 1 wt. %. PEG-120-methylglucose dioleate was not present in C5. Example 15 had 0.5 wt. % PEG-120-methylglucose dioleate, and Example 16 had 1 wt. %. Viscosity increased as the amount of PEG-120-methylglucose dioleate increased with the highest measured viscosity being 16.7 Pas for Example 6 after four weeks at 25° C. Examples 15 and 16 had viscosities ranging from 7.8 Pas to 16.7 Pas, for viscosity measurements after 4 weeks at 25° C. and at 40° C., all of which are within the consumer-acceptable range of 2000 mPa-s to about 20,000 mPa-s. These results show that the compositions of Examples 15 and 16 had suitable viscosities after 4 weeks and at varying temperatures, which emphasizes that PEG-120-methylglucose dioleate can be used in combination with botanical oils and sulfate-free surfactants to provide shampoo compositions having consumer-acceptable viscosities for longer shelf times and varying temperature ranges.

TABLE 7

| | | Ex. | Ex. | Ex. | Ex. | Ex. | Ex. |
|---|---|---|---|---|---|---|---|
| | C5 | 17 | 16 | 17 | 18 | 19 | 20 |
| Alkyl Olefin | 12 | 12 | 12 | 12 | 12 | 12 | 12 |
| Sulfonate ⁽¹⁰⁾ | | | | | | | |
| Cocamidopropyl | 4 | 4 | 4 | 4 | 4 | 4 | 4 |
| Betaine | | | | | | | |
| Cocamide MEA ⁽¹¹⁾ | 2 | 2 | 2 | 2 | 2 | 2 | 2 |
| Coconut oil | 1 | 1 | 1 | 1 | 1 | 1 | 1 |
| Ceteareth-60 Myristyl | 0 | 0.3 | 0.5 | 0.75 | 1 | 1.25 | 1.5 |
| Glycol ⁽¹⁵⁾ | | | | | | | |
| Guar Hydroxypropyl | 0.4 | 0.4 | 0.4 | 0.4 | 0.4 | 0.4 | 0.4 |
| Trimonium Chloride | | | | | | | |
| Hydrogenated Castor | 0.06 | 0.06 | 0.06 | 0.06 | 0.06 | 0.06 | 0.06 |
| Oil | | | | | | | |
| Citric Acid / Sodium | Adjust to pH ~5-7 | | | | | | |
| Citrate Dihydrate / | | | | | | | |
| HCl / NaOH | | | | | | | |
| Water, Fragrance, | Q.S. to 100 | | | | | | |
| Preservatives | | | | | | | |
| Viscosity (Pas- after | 0.5 | 3.1 | 3.7 | 3.9 | 4.9 | 5.7 | 7.1 |
| 4 weeks 25° C.) | | | | | | | |

⁽¹⁵⁾ ELFACOS ® GT 282S

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A shampoo composition comprising:
a) a surfactant system comprising an anionic surfactant;
b) a conditioning agent comprising a botanical oil;
c) about 0.5 wt. % to about 3 wt. % of an associative thickener; and
d) a liquid carrier,
wherein the composition has a viscosity of about 2000 mPa-s to about 20,000 mPa-s, according to the Rheology method.

2. The shampoo composition of claim 1, wherein the associative thickener comprises a hydrophobically modified polyether.

3. The shampoo composition of claim 2, wherein the hydrophobically modified polyether comprises a hydrophobic portion selected from cetyl alcohol, stearyl alcohol, myristyl alcohol, cetearyl alcohol, oleyl alcohol, lauryl alcohol, and combinations thereof, and a hydrophilic portion of repeating ethylene oxide groups with repeat units from 10-300, and combinations thereof.

4. The shampoo composition of claim 2, wherein the hydrophobically modified polyether is selected from PEG-(60-180)-methylglucose dioleate, PEG-(60-180)-methylglucose trioleate, sorbeth (180-240) tetraoleate, PEG-(100-200)-pentaerythrityl tetrastearate, PEG-(60-180)-distearate, PEG-(160-240)-glyceryl stearate, or ceteareth-(60-120)-myristyl glycol.

5. The shampoo composition of claim 2, wherein the hydrophobically modified polyether is selected from Formula I to VII and combinations thereof: 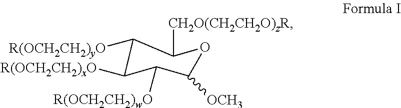
wherein R=H and w+x+y+z=60 to 180; 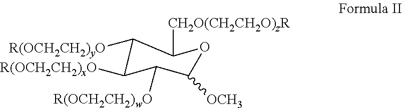
wherein R is H or C₁₇H₃₃CO (Average 3 R group are C₁₇H₃₃CO) and w+x+y+z=60 to 180; 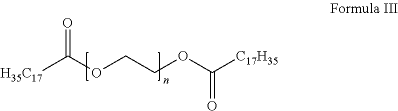
wherein n=60 to 180; 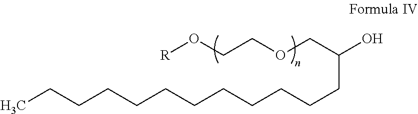
wherein n=60 to 120 and R=Ceteareth; 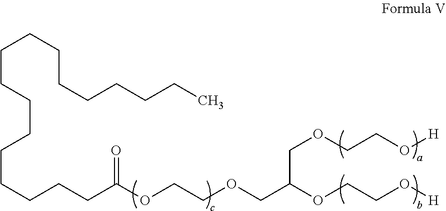
wherein a+b+c=160 to 240; 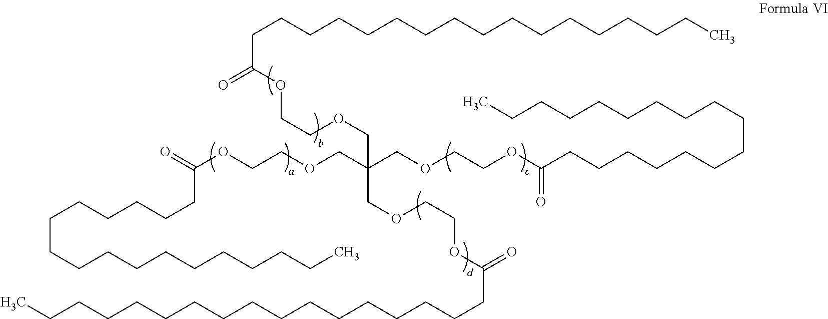
wherein a+b+c+d=100 to 200; and 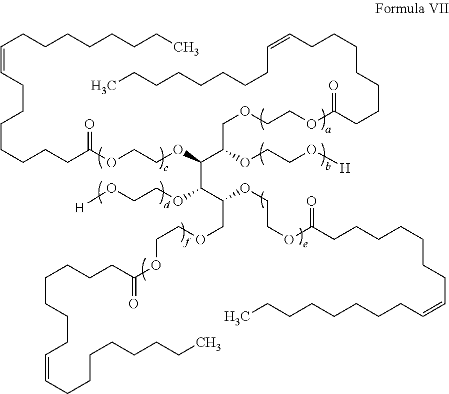
wherein a+b+c+d+e+f=180 to 240.

6. The shampoo composition of claim 1, wherein the botanical oil comprises triglycerides and esters of fatty acids, wherein the fatty acids are either saturated, monounsaturated, or polyunsaturated and contain varying chain lengths ranging from C8 to C30.

7. The shampoo composition of claim 6, wherein the botanical oil is selected from soybean oil, canola oil, safflower oil, argan oil, jojoba oil, coconut oil, shea butter, orange peel wax, tea tree oil, and CCCCCCCVVVVVC bran oil.

8. The shampoo composition of claim 1, wherein the composition comprises about 0.02 wt. % to about 1 wt. % of the botanical oil.

9. The shampoo composition of claim 1, wherein the composition comprises about 10 wt. % to about 25 wt. % of the surfactant system.

10. The shampoo composition of claim 1, wherein the composition is free of sulfated surfactants.

11. The shampoo composition of claim 1, further comprising a co-surfactant selected from alkyl amidopropylbetaine, alkyl betaine, alkanolamide, alkyl hydroxysultaine, alpha olefin sulfonate, alkyl glucoside, and combinations thereof.

12. The shampoo composition of claim 1, wherein the composition is substantially free of silicone.

13. An aqueous shampoo composition, comprising:
a) an anionic surfactant
b) a co-surfactant;
c) a cationic polymer;
d) a botanical oil;
e) an associative thickener, and
f) water,
wherein the shampoo composition provides an average detangling of wet hair of less than 300 gf according to the Hair Texture Analysis Method.

14. The aqueous shampoo composition of claim 13, wherein the botanical oil is selected from soybean oil, canola oil, safflower oil, argan oil, jojoba oil, coconut oil, shea butter, orange peel wax, tea tree oil, and rice bran oil.

15. The aqueous shampoo composition of claim 13, wherein the associative thickener is selected from Formulas I to VII and combinations thereof: 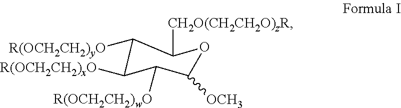
wherein R=H and w+x+y+z=60 to 180; 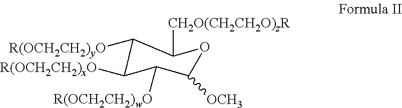
wherein R is H or C₁₇H₃₃CO (Average 3 R group are C₁₇H₃₃CO) and w+x+y+z=60 to 180; 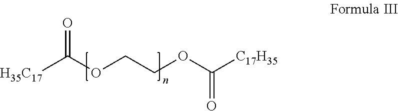
wherein n=60 to 180; 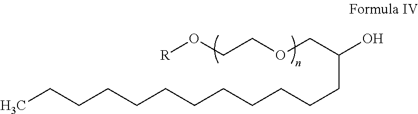
wherein n=60 to 120 and R=Ceteareth; 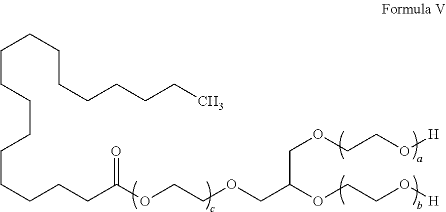
wherein a+b+c=160 to 240; 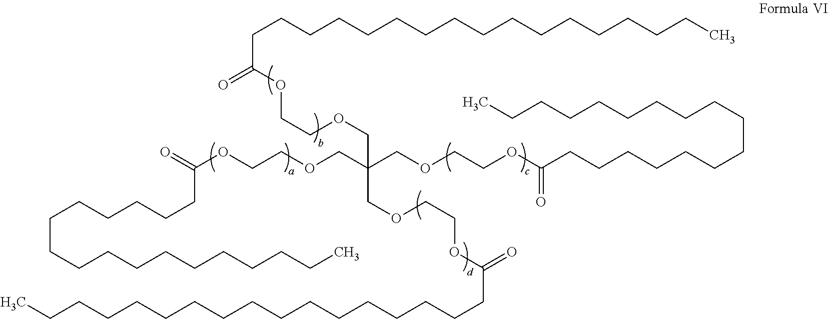
wherein a+b+c+d=100 to 200; and 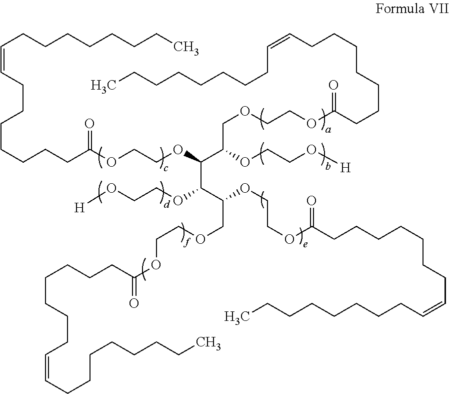
wherein a+b+c+d+e+f=180 to 240.

16. The aqueous shampoo composition of claim 15, wherein the composition comprises about 1 wt. % to about 2 wt. % PEG-(60-180)-methylglucose dioleate.

17. The aqueous shampoo composition of claim 16, wherein the composition is substantially free of silicone.

18. The aqueous shampoo composition of claim 13, wherein the composition comprises about 0.02 wt. % to about 1 wt. % of the botanical oil.

19. An aqueous shampoo composition, comprising:
a) an associative thickener;
b) a botanical oil;
c) a surfactant system comprising an anionic surfactant; and
d) a liquid carrier,
wherein a continuous network is formed throughout the aqueous shampoo composition via cooperation of thickener components that have a hydrophobic character with other components of the composition that have a hydrophobic character.

20. The aqueous shampoo composition of claim 19, wherein the surfactant system comprises cocamidopropyl betaine, cocamide monoethanolamine (MEA), a sulfonate, or a combination thereof.
